(19) United States
(12) Patent Application Publication
(10) Pub. No.: US 2025/0195442 A1
(43) Pub. Date: Jun. 19, 2025

(54) POLYMER-COATED NANOPARTICLES

(71) Applicant: 20Med Therapeutics B.V., Leiden (NL)

(72) Inventors: Hendrika Maria ROELOFS-HAARHUIS, Leiden (NL); Jacob RIP, Leiden (NL)

(73) Assignee: 20Med Therapeutics B.V., Leiden (NL)

(21) Appl. No.: 18/844,473

(22) PCT Filed: Mar. 9, 2023
(86) PCT No.: PCT/NL2023/050113

§ 371 Date: Sep. 6, 2024

(30) Foreign Application Priority Data
Mar. 9, 2022 (NL) 2031209

(51) Int. Cl. A61K 9/51; B82Y 5/00

(52) U.S. Cl. CPC A61K 9/5146; A61K 9/5138; A61K 9/5192; B82Y 5/00

(57) ABSTRACT

The invention is in the field of nanoparticles. In particular, the invention relates to a polymer-coated nanoparticle comprising a biologically active payload. The invention further relates to a method to prepare the polymer-coated nanoparticle. The polymer-coated nanoparticles may be used as a medicament, preferably as a vaccine, such as a prophylactic and/or a therapeutic vaccine.

The invention is in the field of polymeric nanoparticles for medical use. In particular, the invention relates to a polymer-coated nanoparticle comprising a biologically active payload. The invention further relates to a method to prepare the polymer-coated nanoparticle.

Nanostructures, such as polymeric nanoparticles, are used in the medical field as drug and/or gene delivery systems to provide a method for targeted drug delivery and timed release. Several publications that illustrate the suitability thereof are Hoshyar et al. (Nanomedicine (Lond.), 2016, 11 (6), 673-692), Pudlarz and Szemraj (Open Life Sci. 2018:13: 285-298) and Ross et al. (J. Control Release. 2015, 219, 548-559).

As for instance described in the aforementioned article by Pudlarz and Szemraj, these nanoparticles can be made of a wide range of materials including synthetic or natural polymers, lipids or metals and are designed to facilitate penetrance of a payload (e.g. a drug) through physiological barriers. Biologically active payloads such as oligonucleotides, polynucleotides, peptides and proteins in particular have shown great potential. These types of biologically active payloads can advantageously be used because of their high activity, specificity and low toxicity.

A further example of nanoparticles for medical use are described in WO2020/183238.

To fully take advantage of the properties of nanoparticles, the size, shape and surface chemistry typically need to be optimized. It is therefore often required to specifically design the nanoparticle for a particular purpose. Factors that may affect the effectiveness of nanoparticle-based drug delivery systems are i.a. the size of the nanoparticle, the shape of the nanoparticle and the material it is constructed of. The material of the nanoparticle can, for instance, determine the responsiveness to pH changes typically occurring during physiological processes

While there are many advantages associated with nanoparticle-based drug delivery systems, there are also a number of challenges. One particular challenge may be considered the immune reaction it may initiate after the nanoparticles are administrated to a human body, as the human body may consider the nanoparticles to be foreign objects. As a reaction the body typically initiates a fast immune response to eliminate the nanoparticles. The resulting elimination of the nanoparticles is primarily achieved by the mononuclear phagocyte system and the reticuloendothelial system. Due to this rapid recognition and subsequent elimination, the systemic circulation of the nanoparticles and tissue distribution are affected.

Further, in particular cases, for instance for the delivery of negatively charged payloads, the nanoparticle may carry a net positive charge. However, although such positively charged nanoparticles are eminently suitable to bind and condense negatively charged payloads, they are typically unsuitable for intravenous administration, as anionic serum proteins bind to the cationic nanoparticles. The nanoparticles may therefore cause aggregation or lysis of cells in the blood which can cause severe toxicity. Additionally, the high positive charge is likely to hamper the distribution of the nanoparticles after local administration, as the extracellular matrix of local sites is generally negatively charged.

It is an object of the present invention to provide a nanoparticle that at least in part overcomes the above-mentioned drawbacks. The present inventors surprisingly found that this is achieved by a polymer-coated nanoparticle comprising a cationic core and a polymer coating. The polymer coating can advantageously lower the positive charge of the cationic core and lower the cytotoxicity.

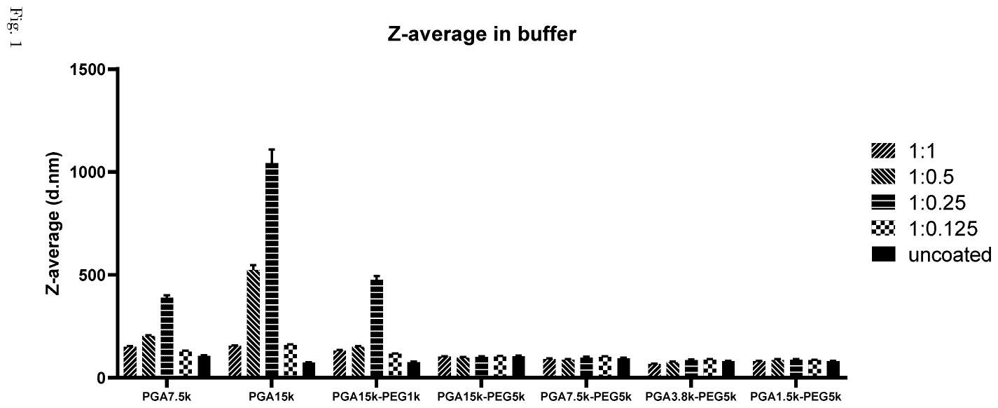

FIG. 1 illustrates the average size of polymer-coated nanoparticles according to the present invention and of comparative nanoparticles.

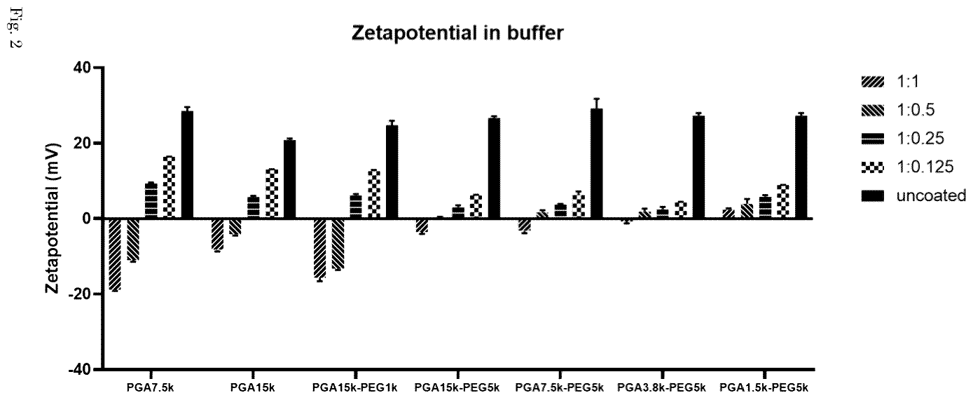

FIG. 2 illustrates the zeta potential of polymer-coated nanoparticles according to the present invention and of comparative nanoparticles.

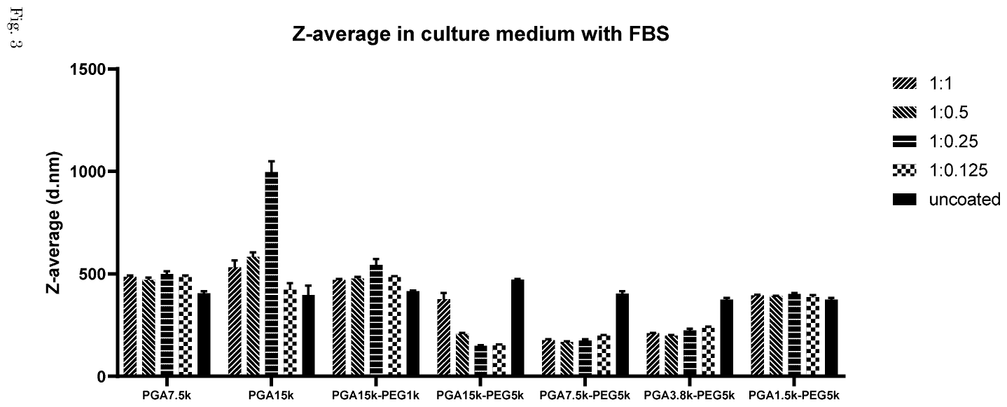

FIG. 3 illustrates the average size of polymer-coated nanoparticles according to the present invention and of comparative cationic nanoparticles in cell culture medium comprising fetal bovine serum.

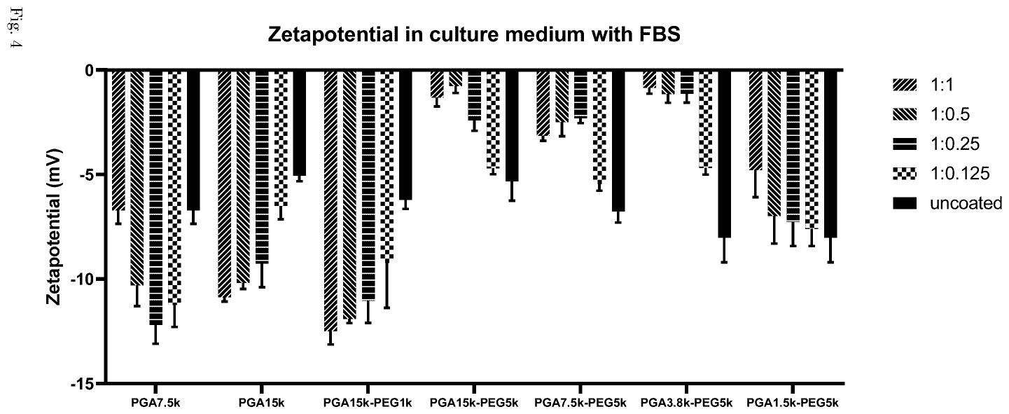

FIG. 4 illustrates the zeta potential of polymer-coated nanoparticles according to the present invention and of comparative nanoparticles in cell culture medium comprising fetal bovine serum.

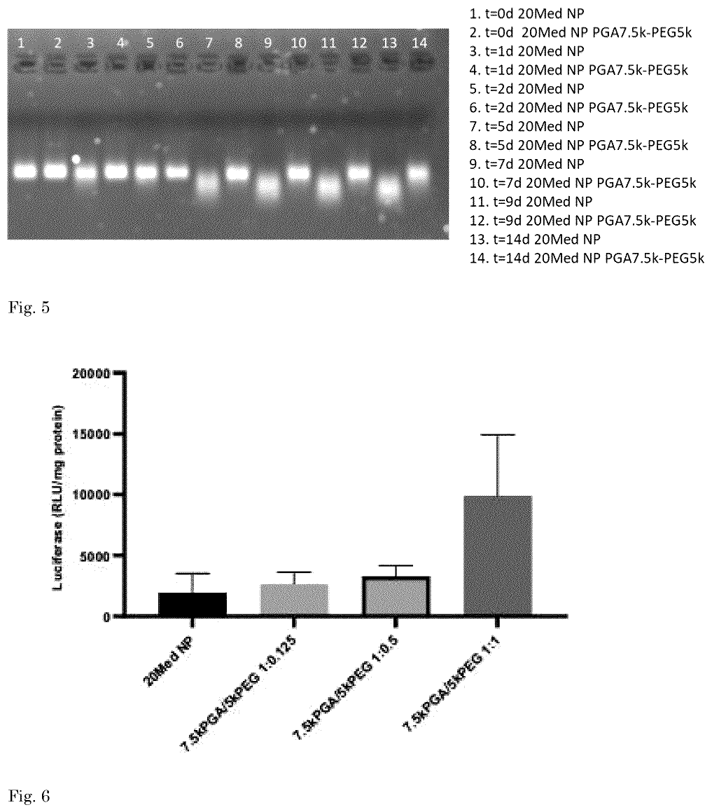

FIG. 5 illustrates the Luciferase activity in muscle homogenates after im. injection with mRNA-luc containing polymer-coated nanoparticles with different coatings and a comparative cationic nanoparticle.

FIG. 6 illustrates the antibody levels after immunization of mice with mRNA encoding SARS-COV-2-spike protein using polymer-coated nanoparticles according to the invention and a comparative cationic nanoparticle as a control.

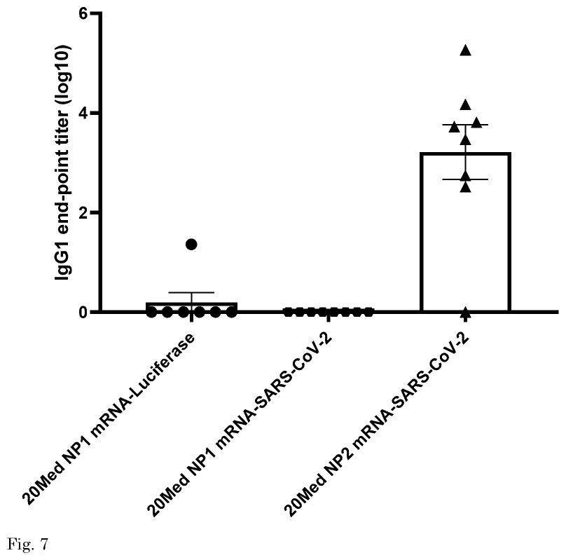

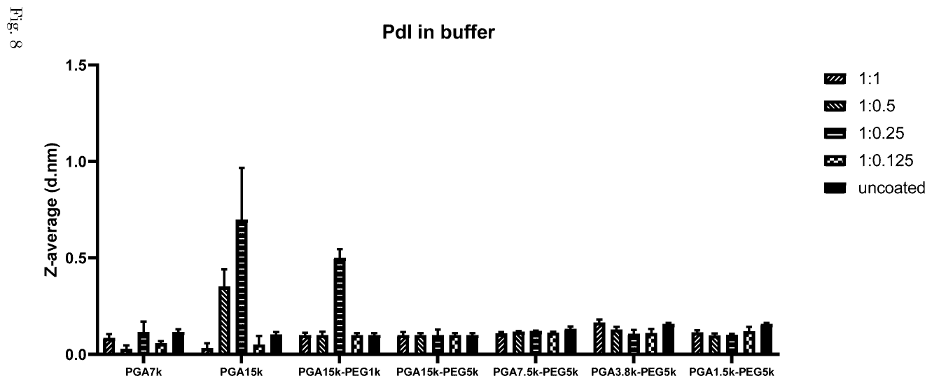

FIG. 7 illustrates the quality of mRNA released from polymer-coated nanoparticles and a comparative cationic nanoparticle after storage at 37° C.

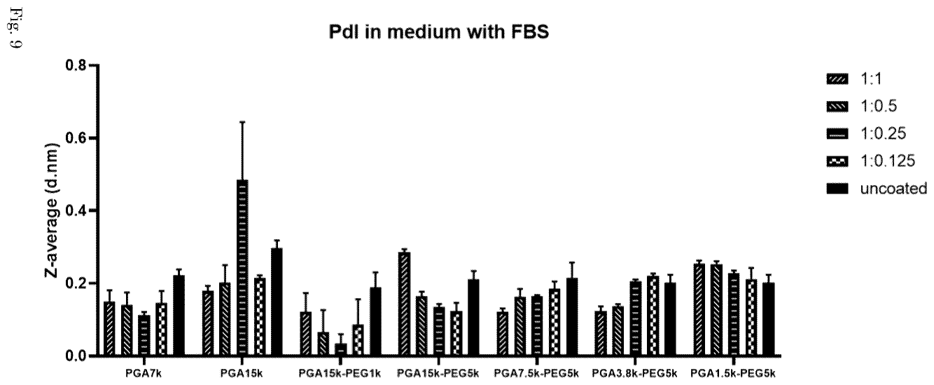

FIG. 8 illustrates the polydispersity index of polymer-coated nanoparticles with different coatings and a comparative cationic nanoparticle.

FIG. 9 illustrates the polydispersity index of polymer-coated nanoparticles with different coatings and a comparative cationic nanoparticle in a cell culture medium comprising fetal bovine serum.

Thus, in a first aspect, the present invention is directed to a polymer-coated nanoparticle comprising a cationic core and a polymer coating. The polymer-coated nanoparticle further comprises a biologically active payload and the polymer coating comprises a block copolymer comprising a polyanionic segment and a neutral segment. The cationic core optionally comprises a poly(amido)amine cationic polymer, a poly(ester amine) cationic polymer and/or a quinoline-functionalized cationic polymer.

The term “segment” as used herein is to be understood as a polymeric structure of any length.

It was surprisingly found that the combination of the neutral and polyanionic segment in the block co-polymer of the coating provides advantageous results. The size of the polymer-coated nanoparticles according to the present invention is more uniformly distributed and smaller than when the neutral segment is not present in a polymer coating. Additionally, the zeta potential of the polymer-coated nanoparticle was generally found to be closer to 0 mV, compared to comparative cationic nanoparticles that are lacking the polymer coating.

The term “neutral segment” is herein used to describe a segment with an overall neutral charge. The neutral segment may accordingly comprise a charge, as long as the charge is counter balanced. An example thereof is a zwitterionic component. Accordingly, the neutral segment may thus comprise a non-charged component and/or a zwitterionic component.

The neutral segment is typically used to shield the cationic core from external factors, such as large macromolecules. As a result, the neutral segment can advantageously allow for decreased in vivo toxicity of the polymer-coated nanoparticles.

It is particularly suitable for the neutral segment to comprise poly(carboxybetaine acrylamide), poly(carboxybetaine methacrylamide), poly(sulfobetaine metacrylate), poly(methacryloyloxyethyl phosphoryl choline, polyvinylpyridiniopropanesulfonate), polyethylene glycol (PEG), polypropylene glycol, poly(glycerols) (PGs), poly(oxazolines) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), polysarcosine, polyacrylamide and poly(N-acryloylmorpholine) (PAcM) or a combination thereof.

Using PEG as nanoparticle material or as a coating (PEGylation) for a nanoparticle is well-known for drug and gene delivery, as i.a. described by Suk et al. (Adv. Drug Deliv Rev. 2016, 1:99, (Pt A): 28-51) and by Thi et al. (Polymers, 2020, 12, 298). PEGylation may be suitably used to improve the efficiency of drug and gene delivery as the PEG may prevent protein absorption and may prevent uptake by the mononuclear phagocytic system. The surface of the nanoparticles can accordingly be shielded from external factors, increasing i.a. systemic circulation time. Additionally, PEG is considered safe for use in humans and is classified as Generally Regarded as Safe (GRAS) by the FDA. Accordingly, it is preferred that the neutral segment comprises polyethylene glycol (PEG).

The block copolymer further comprises a polyanionic segment. Polyanionic segment is used herein to describe a segment that comprises more than one negative charge in the segment. Preferably, the polyanionic segment comprises an oligomer or a polymer, in such cases each of the monomeric building blocks typically carries at least one negative charge. It may be appreciated that the polyanionic segment may be present as a salt e.g. as a sodium salt.

Suitable polyanionic segments comprise polyglutamic acid (PGA) or polyaspartic acid in either the L-, D-enantiomeric form or a racemic mixture, polyacrylic acid or a combination thereof. Moreover, both isoforms of PGA, α-PGA and γ-PGA, can each individually or together be used. α-PGA can be preferred for its degradability, while γ-PGA may be particularly preferred for its particular physiological interaction and/or immunogenic response. Similarly, both isoforms of polyaspartic acid, α- or β-polyaspartic acid, can each individually or together be used. Polyaspartic acid and derivatives may also be obtained from polysuccinimide. PGA, especially the α-L-PGA, is known as a non-immunogenic, biodegradable material, which can be used in the production of nanoparticles as well as for the delivery of active agents. It is accordingly preferred that the polyanionic segment comprises polyglutamic acid, more preferably poly-α-L-glutamic acid.

The neutral and polyanionic segments are present in a block copolymer. Typically, the block copolymer comprises an A-B alternating polyblock copolymer structure. This allows for an optimal positioning of the polymer coating relative to the cationic core (vide infra). Herein, A denotes the one or more neutral segments and B denotes the one or more polyanionic segments. In other words, the polymer coating may thus comprise one or more neutral segments and one or more polyanionic segments. A-B alternating polyblock copolymer is to be understood as a polymer wherein A and B are present in alternating fashion for at least part of the polymer, preferably for essentially the entire polymer. It may be appreciated that this implies that, in case of an uneven number of segments, either more polyanionic segments are present than neutral segments or vice versa. In other words, for an alternating polyblock copolymer comprising 5 segments, it can be A-B-A-B-A or B-A-B-A-B. Preferably, the block-copolymer is an A-B-A tri-block copolymer, a B-A-B tri-block copolymer or an A-B di-block copolymer. An A-B di-block copolymer is most preferred.

The molecular weight of the segments may influence the properties of the polymer-coated nanoparticle. It was found that a weight average molecular weight of above 0.5 kDa for a neutral segment provides a decrease in zeta-potential and more uniform distribution of the average particle size. A lower molecular weight for the neutral segment may result in a lower shielding capability between the cationic core and the environment. It is therefore preferred that in the polymer coating, at least one, preferably each of the neutral segments individually, has a weight average molecular weight of at least 0.5 kDa, preferably at least 2 kDa, more preferably at least 4 kDa. In case that there are two or more neutral segments, each of the segments may thus have either a different or the same weight average molecular weight than the other segment(s). Preferably, each of the neutral segments has the same molecular weight. Particularly good results have been obtained wherein at least one of the neutral segment has a weight average molecular weight of around 5 kDa.

For the polyanionic segment, the weight average molecular weight may be selected to allow for a decrease in zeta potential and a more uniformly distributed average particle size of the polymer-coated nanoparticles. A too low molecular weight may be insufficient to provide good binding of the polymer coating to the cationic core. On the other end, a too high molecular weight may lead to nanoparticle instability and aggregation. Particularly suitable weight average molecular weights for the polyanionic segment are such that that at least one, preferably each of the one or more polyanionic segments individually, has a weight average molecular weight of at least 0.5 kDa, preferably at least 2 kDa, more preferably at least 3 kDa, even more preferably at least 5 kDa, most preferably at least 6 kDa. In the most preferred embodiment, the one or more polyanionic segments individually has a weight average molecular weight of approximately 7.5 kDa. In case of multiple polyanionic segments, each segment may thus either have a different molecular weight or the same molecular weight. Preferably, each of the polyanionic segments has the same molecular weight. More preferably each of the polyanionic segments has the same molecular weight and the same overall charge.

The neutral and polyanionic segments can be present in the polymeric coating in a variety of ratios. The preferred ratio may depend on i.a. the properties of the cationic core including its charge density as well as the charge density of the block copolymer. Typically, the weight ratio of the neutral segment to the polyanionic segment in the block copolymer is between 25:1 and 1:25, preferably between 15:1 and 1:15, more preferably between 4:1 and 1:4, most preferably between 2:1 and 1:2. The weight ratio is based on the weight average molecular weight of the segments.

Particularly good results have been obtained for di-block copolymer comprising PEG and PGA, particularly α-L-PGA. Specifically wherein PEG has a weight average molecular weight of approximately 5 kDa and PGA of approximately 7.5 kDa. Accordingly, the most preferred polymer coating comprises PGA7.5 kDa-PEG5 kDa.

Typically, the polymer coating allows for polymer-coated nanoparticles with an average size and zeta potential within a certain range. Both parameters and suitable measuring methods are known in the art. A suitable technique for measuring the average size is dynamic light scattering (DLS) and for the zeta-potential electrophoretic light scattering (ELS). The measurements can be performed in a buffered aqueous solution at a physiologically relevant pH.

Aside from the pH, other factors that may include the measured data include the presence of macromolecules and/or serum proteins e.g. fetal bovine serum (FBS) or any other serum proteins known to the skilled person as well as other buffer components such as salts. These and other compounds may be suitably used to mimic physiological conditions. Typically, a solution of 20 mM HEPES (N-(2-hydroxyethyl) piperazine-N′-(2-ethanesulfonic acid)) buffered culture medium (DMEM) with 10% (v/v) FBS is used as serum proteins for the measurements. In such cases, the average size may be larger and the zeta-potential may be lower compared to the properties measured in only a buffer solution. A factor of influence in the measurement of nanoparticles is that each sample contains a population of particles with varying sizes or multiple populations that differ significantly in size. The heterogeneity in size of a population may be measured using DLS and expressed using the Polydispersity index (PDI). In general, samples with a PDI of <0.30 can be considered monodisperse while higher numbers have a larger spread in their particle size or consist of multiple populations. Ideally a nanoparticulate system is monodisperse.

It was surprisingly found that the PDI is low for the polymer-coated nanoparticles. The PDI may be less than 0.2, more preferably less than 0.15, when measured in a buffer solution. Advantageously, the PDI index of the polymer-coated nanoparticles was found to be low in a buffer comprising serum proteins. This is advantageous as this indicates that little agglomeration of the nanoparticles occurs. Accordingly, the PDI may be less than 0.2, typically less than 0.15 when measured in the presence of serum proteins. The polymer-coated nanoparticles may thus be considered monodisperse. The low PDI is in particular for a PGA-PEG coating wherein the PGA has an weight average molecular weight between 3 kDa and 15 kDa and wherein PEG has a weight average molecular weight of around 5 kDa.

The PDI may also be compared to a comparative cationic nanoparticles (i.e. having the same cationic core) that are free of the polymeric coating. In such cases the PDI of the polymer-coated nanoparticles may be at least 0.05 less than the polydispersity index of the comparative nanoparticle, for instance when measured in the presence of serum proteins, such as in a solution of 20 mM HEPES buffered culture medium (DMEM) with 10% (v/v) FBS.

The average size and polydispersity index referred herein are measured in accordance with ISO 22412:2017. More specifically, they can be measured on a Zetasizer Nano ZS from Malvern (Kassel, Germany) at a fixed angle of 173° backscatter. The average of three measurements can accordingly be taken. The settings may be measured in 20 mM HEPES with a temperature of 25° C., viscosity of 0.94 cP (i.e. 0.94 mPa·s), reflection index (RI) 1.330, with a dielectric constant of 78.3. As a measurement control polystyrene particles of e.g. 100 nm can be used, such as Nanosphere™ Size Standards from ThermoFisher Scientific.

The size of the polymer-coated nanoparticle is preferably sufficiently low to provide a sufficient cellular uptake. The cellular uptake is generally increased by a smaller size as this allows for uptake in cells by different (receptor-mediated) endocytosis pathways. These pathways are typically not available for larger particles. The polymer-coated nanoparticles may therefore have an average size of at most 350 nm, preferably at most 200 nm, more preferably at most 100 nm, as determined in accordance with ISO 22412:2017 in a buffer, in the absence of serum proteins.

In the presence of serum proteins, e.g. FBS, the average size is typically larger. Typically, the polymer-coated nanoparticle has an average size of at most 350 nm, preferably at most 200 nm, in presence of serum proteins, as determined by ISO 22412:2017.

The average size of the nanoparticles according to the present invention can be compared to a comparative cationic nanoparticle that is free from the polymer coating in the presence of these serum proteins. It was found that, especially for a PGA-PEG coating wherein the PGA has an weight average molecular weight between 3 kDa and 15 kDa and wherein PEG has a weight average molecular weight of around 5 kDa, that the average size of the nanoparticles according to the present invention is significantly smaller than the average size of the comparative nanoparticles. Preferably, the average size of the polymer-coated nanoparticle in presence of serum proteins is at least 200 nm less, preferably at least 250 nm less, more preferably at least 300 nm less than the size of a comparative cationic nanoparticle that is free from the polymer coating.

The zeta-potential of the polymer-coated nanoparticles is preferably sufficiently low to reduce the risks of aggregation of blood cells and thus toxicity. Furthermore, a near neutral surface charge typically allows for increased distribution of the polymer-coated nanoparticles after local administration, as it may not bind as strongly to negatively charged components in the extracellular matrix. The clearance rate from the circulation by the filtering organs like liver, spleen and kidneys, of polymer-coated nanoparticles with near neutral surface charge, is typically also reduced. Sufficiently low zeta potentials are typically between-10 and 20 mV, preferably between-5 and 10 mV, more preferably between 0 and 10 mV, as determined by ISO 13099-1.

Zetapotential as referred herein is determined in accordance with ISO 13099-1. More specifically, for the measurement of zetapotential electrophoretic light scattering (ELS) with a Zetasizer Nano ZS from Malvern can be used. A folded capillary cell (DTS1070) with nanoparticle samples can be diluted in 20 mM HEPES. The dispersant for measurement can be 20 mM HEPES, the temperature 25.0° C., the viscosity 0.94 cP, the RI:1.330, and the dielectric constant 78.3. An average of three measurements can be taken to find the zetapotential

The zeta-potential of the nanoparticles according to the present invention can be compared to a comparative cationic nanoparticle that is free from the polymer coating. In such cases, the zeta-potential of the polymer-coated nanoparticle is typically at least 10 mV, preferably at least 20 mV, more preferably at least 25 mV lower than the zeta potential of the comparative nanoparticle.

Additionally, the zeta-potential of the nanoparticles according to the present invention may be compared with a comparative cationic nanoparticle that is free from the polymer coating in the presence of serum proteins, e.g. FBS. In such case, it was found that the zeta-potential of the nanoparticles according to the present invention is less negative, i.e. closer to 0 mV, than the zeta-potential of a comparative nanoparticle. This is particularly the case for a PGA-PEG coating, wherein the PGA has a weight average molecular weight between 3 kDa and 15 kDa and wherein the PEG has a weight average molecular weight of approximately 5 kDa.

Especially the combination of the lower zeta-potential, the average size and the polydispersity index of the polymer-coated nanoparticles of the present invention, lead to an advantageous nanoparticle for medical use. The most advantageous properties were found for a PGA7.5 kDa-PEG5 kDa coating.

The charge of the cationic core is thus at least partially counterbalanced by the polymer coating. The cationic core comprises a cationic polymer. The cationic core may also be referred to as a cationic polymeric core. For instance, the polymers as described by WO 2012/165953, which is incorporated herein by reference, may be suitably used for the cationic core. WO 2012/165953 describes a polyamido-amine (PAA) polymer that may be used to form nanoparticles that can function as carriers for biologically active components. Preferably, the cationic polymer is a poly(amido)amine, a poly(ester amine) and/or a quinoline-functionalized cationic polymer. These polymers, in combination with the polymer coating result in surprisingly good properties of the nanoparticle, including a zeta-potential close to 0 mV, a small average nanoparticle size and a low PDI. Additionally, it is beneficial that the suitable cationic polymers are biodegradable.

Quinoline-functionalized cationic polymers suitable for the present invention comprises a polyamine segment of formula (I),

[X-B]ₙ-[X-Q]ₘ- (I)

wherein X is based on a bis(α,β-unsaturated carbonyl) monomer, B is based on an amine monomer, Q is based on a quinoline-containing amine monomer comprising a quinoline moiety, and wherein m is 1 or more, and n is 0 or more, preferably 1 or more.

Again, the term “segment” as used herein is to be understood as a polymeric structure of any length. The polyamine segment according to formula (I) is based on bis(α,β-unsaturated carbonyl) monomer (X) and amine monomers (Q and B), a combination of which result in a repeating unit. In each repeating unit may thus be present either a Q amine (i.e. [X-Q]) or a B amine (i.e. [X-B]), which can randomly be positioned in the polyamine segment. The amine monomer B does not comprise a quinoline moiety and differs in at least this aspect from the quinoline-containing amine monomer Q. The present polyamine segment can thus be regarded as a copolymer, in particular a random co-polymer.

The labels n and m represent the number of each repeating unit in the polyamine. In the polyamine, Q is always present, thus m equals 1 or more, while B is optionally present: n equals 0 or more, but preferably 1 or more.

In typical embodiments, the sum of n and m is in the range of 2-250, preferably 5 to 100. The polyamine has a typical weight average molecular weight in the range of 1 to 100 kD, preferably in the range of 5 to 40 kD.

The relative amount of Q vis-à-vis B can be expressed as the ratio m to n. As the amine monomer B is optionally present, n may be zero and the ratio may accordingly be 1:0, i.e. effectively 1. However, preferably the polyamine segment and/or the polymer contains more B than Q. In particular embodiments, the ratio m to n is in the range of 1:20 or more (i.e. 0.05 or more, e.g. 0.05 to 20). Preferably, the ratio m to n is in the range of 1:10 to 10:1, more preferably in the range of 1:5 to 2:1, most preferably in the range of 1:4 to 1:1 such as about 1:3, as particular good results were obtained in that range.

In particular preferred embodiments, the polyamine segment, preferably said quinoline-functionalized cationic polymer, has a structure according to formula (II).

{[X-B]ₙ-[X-Q]ₘ}_{q}-T (II)

In further preferred embodiments, the polyamine segment and preferably the quinoline-functionalized cationic polymer for use in the present invention has a structure according to any of formulae (IIIa), (IIIb), (IIIc), (IIId), and combinations thereof, wherein n, m, and p can be as defined for formula (I).

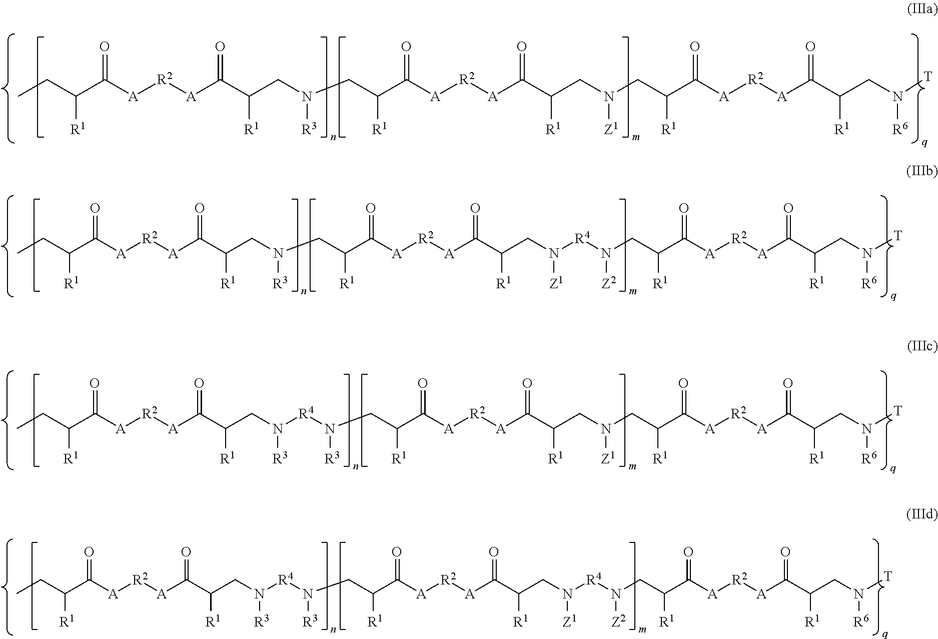

wherein A is selected from the group consisting of C(R¹)₂, O, N(R¹), S, and combinations thereof, preferably N(R¹)
R¹ is selected from the group consisting of H, halides and C₁-C₄ alkyls, which are optionally substituted with one or more halides, and combinations thereof;
R² is selected from the group consisting of C₁-C₄₀ linear, cyclic and branched hydrocarbylenes, which are optionally substituted and/or interrupted with one or more heteroatoms, and combinations thereof, preferably wherein R² comprises a disulfide moiety, more preferably wherein R² is selected from the group consisting of C₁-C₁₀ hydrocarbylenes, preferably linear alkylenes, interrupted with at least one disulfide moiety, and combinations thereof;
R³ is independently selected from the group consisting of H, C₁-C₄₀ linear, cyclic and branched hydrocarbyls, which are optionally substituted and/or interrupted with one or more heteroatoms, and combinations thereof, preferably wherein R³ comprises a hydroxy moiety, more preferably wherein
R³ is selected from the group consisting of C₁-C₁₀ hydrocarbyl hydroxide, preferably linear alkyl C₁-C₁₀ hydroxide, and combinations thereof;
R⁴ is selected from the group consisting of C₁-C₄₀ linear, cyclic and branched hydrocarbylenes, which are optionally substituted and/or interrupted with one or more heteroatoms, and combinations thereof;
Z¹ represents a group comprising the quinoline moiety;
Z² represents a group comprising the quinoline moiety or is selected from the group consisting of H, C₁-C₄₀ linear, cyclic and branched hydrocarbyls, which are optionally substituted and/or interrupted with one or more heteroatoms, and combinations thereof.

Preferably, Z¹ and optionally Z² independently represent a moiety of formula (I_{Z1}), (I_{Z2}) or (I_{Z3}), preferably a moiety of formula (I_{Z2}),

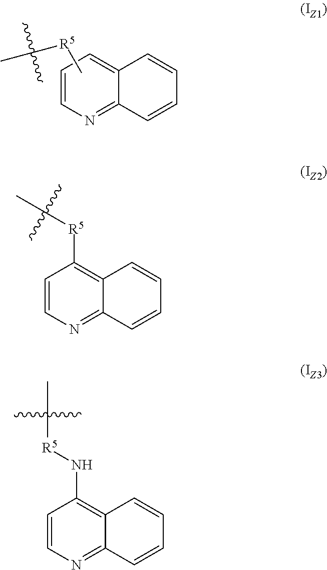

wherein R⁵ is selected from the group consisting of C₁-C₄₀, preferably C₁-C₁₀, more preferably C₁-C₆ linear, cyclic and branched hydrocarbylenes, which formulae are optionally substituted and/or interrupted with one or more heteroatoms, and combinations thereof.

The quinoline moiety may optionally be substituted with one or more substituents, preferably selected from the group consisting of halides, alkyls and alkoxides, both which groups may again be optionally substituted with one or more heteroatoms. In a particularly preferred embodiment, the quinoline moiety comprises a substituent on the 7-position and Z¹ and optionally Z² independently represent a moiety of formula (I_{Z4})

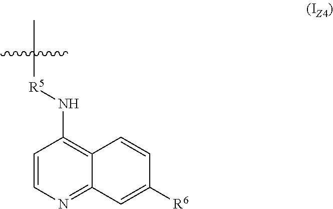

wherein R⁶ is selected from the group consisting of halides, alkyls, alkoxides and mixtures thereof, optionally substituted with one or more heteroatoms, preferably halides, more preferably wherein R⁶ is Cl.

The spacer R⁵ can be of a relatively simple structure, for example a linear unsubstituted alkylene such as butylene (C₄H₈), hexylene (C₆H₁₂) or octylene (C₈H₁₆) while retaining the advantageous effect of improving transfection. Thus, although it is possible to use a spacer that comprises a quinuclidine such as present in the natural product quinine, this is not required.

Further, in formulae (II) and (IIIa)-(IIId), T represents a core of the quinoline-functionalized cationic polymer that preferably has an weight average molecular weight Mw of about 60 to about 25000; q is in the range of 1 to 64; and R⁶ is selected from the group consisting of H, C₁-C₄₀ linear, cyclic and branched hydrocarbyls, which are optionally substituted and/or interrupted with one or more heteroatoms, and combinations thereof.

The core T of the quinoline-functionalized cationic polymer is generally based on a structure with one or more primary and/or secondary amines. These amines are represented in formulae (IIIa)-(IIId) with the group NR⁶. Particularly suitable cores of the quinoline-functionalized cationic polymer include oligo- or polyamine cores such as 1,2-ethylenediamine, tris-(2-aminoethyl)amine as well as polymeric cores bearing primary and/or secondary amines such as polyethyleneimines (PEI), poly(amido amine) polymers or dendrimers (PAMAM), polypropylene imines (PPI), poly(ester amine) polymers (PEAN) and poly(ether amine) polymers (PEAC) and other polymers as described in WO 2012/165953 (referred therein a POL). PEI is particularly preferred as the core and most preferred is PEI 800 with on average 6 primary amines in this respect, as this gave particular good results.

It may be appreciated that the most preferred embodiment is a polymer-coated nanoparticle comprising a cationic core comprising a poly(amido)amine cationic polymer, a poly(ester amine) cationic polymer and/or a quinoline-functionalized cationic polymer. Additionally, in this most preferred embodiment, the polymer coating comprises a PGA-PEG coating, wherein the PGA has an approximate weight average molecular weight of 7.5 kDa and the PEG has an approximate weight average molecular weight of 5 kDa. Further, the ratio of the cationic polymer to the polymeric coating is preferably around 1:1 by weight. This ratio may also be expressed as a molar ratio of the cationic polymer to the polymer coating (N/A ratio). For the most preferred embodiment, a 1:1 weight ratio would be identical to a N/A ratio of approximately 0.6 (e.g. PGA-PEG has 1 charge per ca. 250 Da, the cationic polymer 1 charge per ca. 400 Da, then at 1:1 w/w, this would result in a N/A ratio of 1/1.6=0.625).

For the embodiments wherein the nanoparticle's cationic core is formed by a cationic polymer, at least part of the polyanionic segment is at least partially penetrating the cationic core. Additionally, or alternatively, at least part of the neutral segment is at least partially protruding from the cationic core. Due to the electrostatic attraction between the cationic polymer and the polyanionic segment, these will typically be in close proximity. The core may be visualized as a nanosized sphere formed by the cationic polymer, wherein the polyanionic segment may be placed such that at least part of the segment at least partially penetrates this sphere. The penetration can thus be very minimal, as long as it is enough for the coating to remain electrostatically attached to the cationic nanoparticle. Additionally, or alternatively, at least part of the neutral segment is partially protruding from the core. This protrusion can accordingly also be minimal, as long as it is sufficient to shield the cationic core. For instance, for an A-B block copolymer, the polymer coating may be present as linear chains wherein at least part of the polyanionic segment is partially penetrating the core, and the neutral segment is at least partially pointing away from the core. For an A-B-A tri-block copolymer, the polyanionic segment (B) may, in that case, be closest to the cationic nanoparticle, whereas both neutral segments (A) are deflected from the cationic nanoparticle. In contrast, a B-A-B tri-block polymer will form a loop-like or flower-like arrangement wherein both polyanionic segments (B) interact with the cationic core while the neutral segment (A) will form an extended loop between two anchoring polyanionic segments.

The polymer-coated nanoparticle further comprises a biologically active payload. In general, the biological active payload is embedded within the cationic core. The biological active payload may be any material or combination of materials that can induce a biological or physical response in the human body directly or indirectly. For instance, small molecule drugs are considered biologically active payloads. A particularly suitable biologically active payload comprises oligonucleotides, polynucleotides, oligopeptides, polypeptides, proteins, small molecules or a combination thereof. These for instance include all types of RNA and DNA and their derivatives, i.a. plasmid DNA, dbDNA, hpDNA, c3DNA, minicircles, phosphorodiamidate morpholine oligomers (PMOs), siRNA, mRNA, endless RNA, circular RNA single and/or double stranded RNA including designed guide RNAs (gRNA or sgRNA) used in gene editing techniques. The origin of these materials is irrelevant, thus any artificially constructed or chemically modified oligonucleotides, polynucleotides, oligopeptides, polypeptides and/or proteins are also to be understood to be included by the term biologically active payload. One or more gene constructs are therefore also to be considered a biologically active payload. Moreover, the polynucleotide, oligonucleotide, oligopeptide, polypeptide and/or protein may be based on natural-occurring building blocks, but also on non-natural occurring building blocks such as non-natural nucleosides or non-natural amino acids. Although the present nanoparticular are particularly suitable for polymeric and oligomeric biologically active components, which are accordingly preferred, the biologically active payload may also be non-polymeric and non-oligomeric and may include any active pharmaceutical ingredient.

It may be appreciated that due to the arrangement of the polymer coating relative to the cationic core, the coating adheres to the cationic core and minimizes the exposure of the payload and the cationic core to the environment. The polymer-coated nanoparticle is therefore considered suitable for use as a drug delivery system.

As such, the polymer-coated nanoparticle according to the present invention is particularly suitable for use as a medicament and/or in a medical treatment. The term medical treatment herein includes curative treatments, preventive treatments, prophylactic treatments, diagnostic treatments, and the like. Any way and/or route of administration is understood to be included in the term medical treatment.

More particularly, the polymer-coated nanoparticle is preferably for use as a vaccine. The polymer-coated nanoparticle is surprisingly advantageous for the effectiveness of vaccines, in particularly in vivo. The use of the polymer-coated nanoparticle results in a high protein expression and a high immune response. The polymer-coated nanoparticle is more preferable for use as a prophylactic vaccine and/or for use as a therapeutic vaccine. The most preferred administration method for a vaccine is typically parenteral administration. Parenteral administration includes injection, such as subcutaneous, intradermal, intralymphatic, intravenous, intraocular, intraarticular or intramuscular injection. Similar administration with the same aim can also be used. Other methods, such as oral administration, intranasal administration, inhalation, topical administration, needle-free injection devices or microneedle devices are thus also to be included. Preferably, the polymer-coated nanoparticle is for use as a vaccine by injection, e.g. by intramuscular injection.

Additionally, it was surprisingly found that a higher antigen expression is obtained if the nanoparticles according to the present invention is present in the vaccine. The higher antigen expression is related to an improved effectiveness of the vaccine and thus a better immune response. Additionally, the nanoparticles according to the present invention may act as an adjuvant in a vaccine.

The invention is further directed to a method for forming the polymer-coated nanoparticle. The method comprises combining the block copolymer, the payload and the cationic core in a liquid, wherein the cationic core and the block copolymer are combined in a weight ratio smaller than 1:0.125, such as between approximately 1:0.5 and 1:1, or such as between approximately 1:0.25 and 1:0.5. The preferred ratio may however depend on i.a. the properties of the cationic core including its charge density as well as the charge density of the block copolymer.

As different block copolymers may have varying charge densities, it is preferred to match the ratio of the negative charges of the block copolymer with the positive charges on the cationic core when comparing different block copolymers for coating applications. Similarly, when the cationic core is varied, it is likewise preferred to match the ratio of negative to positive charge in order to compare the different materials. A ratio of positive to negative charge between 1:0.1 up to 1:1 is preferably used.

The ratio between the cationic core, such as a cationic polymer, and the block copolymer may also be expressed as a molar ratio of the cation to anion (herein also referred to as the N/A ratio, vide infra). Typically, the N/A ratio is between 0.1 and 15, preferably 0.2 to 10, more preferably 0.2 to 5, even more preferably between 0.2 and 1, such as between 0.2 and 0.8. In the most preferable embodiment, the N/A ratio is approximately 0.6.

The cationic core is typically primarily based on the cationic polymer. The payload, the block copolymer and the cationic core may be added simultaneously. Generally this results in the payload being at least partially encompassed by the cationic core. The block-copolymer may position relative to the cationic core as described above.

Alternatively, the payload and the cationic core may be added simultaneously, to allow for the payload to be at least partially encompassed by the cationic core. This is followed by adding the block copolymer, to allow the polymeric coating to be positioned relative to the cationic core as described herein above.

Another alternative is that the coating and the cationic core may be added simultaneously, to allow for the coating to position relative to the cationic core as described herein above. This is followed by adding the payload, to allow the payload to be at least partially encompassed by the polymer-coated cationic core.

The liquid wherein the components are added may be any suitable liquid. Particularly preferred liquid is an aqueous buffer solution, such as the commercially available HEPES.

Advantageously, the coating improves stability of the nanoparticles.

As used herein, the singular forms “a”, “an” and “the” are intended to include the plural forms as well, unless the context clearly indicates otherwise. The term “and/or” includes any and all combinations of one or more of the associated listed items. It will be understood that the terms “comprises” and/or “comprising” specify the presence of stated features but do not preclude the presence or addition of one or more other features.

For the purpose of clarity and a concise description, features are described herein as part of the same or separate embodiments, however, it may be appreciated that the scope of the invention may include embodiments having combination of all or some of the features described.

The invention can further be illustrated by the following non-limiting examples.

### Example 1—Polymer-Coated Nanoparticles with pDNA or mRNA

A solution was made of 120 ng/μL pDNA or mRNA encoding eGFP. A cationic polymer was dissolved at a concentration of 3 mg/mL in 20 mM HEPES pH 7.2. The 120 ng/μL solution of pDNA or mRNA was added 1:1 (v/v) to the cationic polymer. The solution was incubated for at least 15 min at room temperature.

The coating material was dissolved in a concentration of 1.5 mg/mL in 20 mM HEPES. The coating was added to the loaded cationic core 1:1 (v/v). The cationic core was coated in a ratio w/w core/coating 1:1; 1:0.5; 1:0.25 and 1:0.125 with the coating material to yield a polymer-coated nanoparticle. The used coatings are provided in Table 1.

TABLE 1
overview coatings

| | Weight average |
|---|---|
| Coating | molecular weight (kDa) |
| PGA15k | 15 |
| PGA 7.5 | 7.5 |
| PEG1k-PGA15k | 16 |
| PEG5k-PGA15k | 20 |
| PEG5k-PGA7.5k | 13 |
| PEG5k-PGA3.8k | 8.8 |
| PEG5k-PGA1.5k | 6.5 |

The ratio w/w core/coating of 1:1; 1:0.5; 1:0.25 and 1:0.125 may also be expressed as the N/A ratio for each of the coatings as provided in Table 2.

TABLE 2
conversion w/w ratio to N/A ratio

| w/w | N/A | | | | | | |
|---|---|---|---|---|---|---|---|
| Polymer/ | | | PGA15- | PGA15k- | PGA7.5k- | PGA3.5k- | PGA1.5k- |
| coating | PGA7k | PGA15k | PEG1k | PEG5k | PEG5k | PEG5k | PEG5k |
| 1:1 | 0.4 | 0.4 | 0.4 | 0.5 | 0.6 | 0.9 | 1.6 |
| 1:0.5 | 0.8 | 0.8 | 0.8 | 1.0 | 1.3 | 1.8 | 3.3 |
| 1:0.25 | 1.5 | 1.5 | 1.6 | 2.0 | 2.5 | 3.7 | 6.6 |
| 1:0.125 | 3.0 | 3.0 | 3.2 | 4.0 | 5.1 | 7.4 | 13.2 |

### Example 2—Average Particle Size and Zeta-Potential of Polymer-Coated Nanoparticles

Cationic cores loaded with pDNA were coated at various w/w ratios with a PEG1k-PGA15k, PEG5k-PGA1.5k, PEG5k-PGA3.8, PEG5k-PGA7.5 or a PEG5k-PGA15k polymer coating to yield a polymer-coated nanoparticle. The average size and zeta potential were measured in 20 mM HEPES pH 7.2 and illustrated in FIG. 1 and FIG. 2. FIGS. 1 and 2 further illustrate the data of a comparative cationic nanoparticle free of a polymer coating.

### Example 3—Comparative Example of Average Particle Size and Zeta-Potential of Coated Nanoparticles without a Neutral Segment

Cationic cores loaded with pDNA, similar to Example 2, were coated with a PGA7.5k or a PGA15k coating. The average size and zeta potential were measured in 20 mM HEPES pH 7.2 and illustrated in FIG. 1 and FIG. 2.

### Example 4—Average Particle Size and Zeta-Potential of Polymer-Coated Nanoparticles Medium with Fetal Bovine Serum (FBS)

Cationic cores loaded with pDNA were coated at various w/w ratios with a PEG1k-PGA15k, PEG5k-PGA1.5, PEG5k-PGA3.8, PEG5k-PGA7.5 or a PEG5k-PGA15k polymer coating to yield polymer-coated nanoparticles. The average size and zeta potential were measured after 30 minutes incubation in 20 mM HEPES buffered culture medium (DMEM) with 10% (v/v) FBS. The results are illustrated in FIG. 3 and FIG. 4. FIGS. 3 and 4 further illustrate the data of a comparative cationic nanoparticle free of a polymer coating.

### Example 5-Comparative Example of Average Particle Size and Zeta-Potential of Coated Nanoparticles in Medium with Fetal Bovine Serum (FBS) without a Neutral Segment

Cationic cores loaded with pDNA, similar to Example 4, were coated at various w/w ratios with a PGA7.5k or a PGA15k coating polymer coating to yield polymer-coated nanoparticles. The average size and zeta potential were measured after 30 minutes incubation in 20 mM HEPES buffered culture medium (DMEM) with 10% (v/v) FBS. The results are illustrated in FIG. 3 and FIG. 4.

### Example 6 Comparative Example of mRNA Stability in Coated Nanoparticles Stored in Buffer at 37° C.

Cationic cores loaded with mRNA were coated in a 1:1 w/w ratio with PGA7.5k-PEG5k similar to Example 1. The polymer-coated nanoparticles were stored in buffer 10 mM Histidine pH 6.5 at 37° C. mRNA intactness in the samples was analyzed using agarose gel electrophoresis and illustrated in FIG. 5.

### Example 7—In Vivo Studies-Luciferase

BALB/c mice were dosed with 10 μg mRNA Firefly Luciferase loaded in polymer-coated nanoparticles having a cationic core of a quinoline-functionalized poly(amido)amine using a 1:25 w/w loading ratio. The different nanoparticle/polymer-coating ratios are illustrated in Table 3.

Fifty μL of the formulations was injected in the bicep femoris in both legs.

TABLE 3
overview treatment groups

| (n) | Coating (w/w ratio) |
|---|---|
| 5 | uncoated |
| 5 | PEG5k-PGA7.5k (1:0.125) |
| 5 | PEG5k-PGA7.5k (1:0.5) |
| 5 | PEG5k-PGA7.5k (1:1) |

Samples of the injected muscles were collected after 24 hours. Muscle homogenates were made by adding 300 μl lysis buffer (Luciferase cell culture lysis buffer) to 100 mg muscle tissue samples. The samples were homogenized with a Beadbug microtube homogenizer for 90 seconds, 4000 rpm at 4° C. After homogenization, the samples were centrifuged 10 min at 10.000 g at 4° C. The obtained supernatant was used for further analysis of the luciferase activity. Bio-Glo™ Reagent (Promega) was used to quantify the luciferase activity in the supernatant of the muscle homogenates. Total protein was measured in the supernatant using a BCA protein assay (Pierce™). The luciferase activity was expressed as relative luminescent units (RLU) per mg protein in the samples.

The Luciferase activity in muscle homogenates after intramuscular injection with mRNA-luciferase containing polymer-coated nanoparticles with different PGA-PEG coatings and a comparative cationic nanoparticle without polymer coating (20Med NP) is illustrated in FIG. 6.

### Example 8—In Vivo Studies SARS-COV-2

Nanoparticles were loaded with mRNA encoding Sars-COV-2 spike protein. A solution was made of 120 ng/μL mRNA encoding SARS-COV-2 spike protein. A cationic polymer was dissolved at a concentration of 3 mg/mL in 10% w/v trehalose, 10 mM Histidine pH 6.5. The 120 ng/μL solution of pDNA or mRNA was added 1:1 (v/v) to the cationic polymer. The solution was incubated for 15 min at room temperature. The following formulations of polymer-coated and nanoparticles without coating were prepared.
Nanoparticle 1 (NP1) consisting of a poly(amido)amine, (250 μg) in 10% trehalose, 10 mM histidine pH 6.5.
Polymer-coated nanoparticle (NP2) consisting of the same poly(amido)amine, 250 μg and a mPEG5k-b-PGA50 coating (250 μg) in 10% trehalose, 10 mM histidine pH 6.5

Mice (CB6F1 (Balb/c×C57BL/6, female 7-9 weeks) were administrated with a vaccine according to Table 4. Blood samples were taken a week before the first immunization, right before the second immunization and 3 weeks after the second immunization.

TABLE 4
Overview vaccines (im = intramuscular injection).

| Group | (n) | Vaccine | 1^{st} immunization | 2^{nd} immunization |
|---|---|---|---|---|
| A | 8 | mRNA | 30 μl im. right leg | 30 μl im left leg |
| | | Luciferase NP1 | | |
| D | 8 | mRNA Antigen | 30 μl im. right leg | 30 μl im left leg |
| | | NP1 | | |
| E | 8 | mRNA Antigen | 30 μl im. right leg | 30 μl im left leg |
| | | NP2 | | |

The IgG1 levels were determined for the individual mice by ELISA, the plates were coated with the whole Spike protein. As can be seen from FIG. 7, immunization with Spike mRNA/NP2 induced antigen-specific IgG1 antibodies. In contrast, low levels of antigen-specific IgG1 antibodies were induced after immunization with Spike mRNA/NP1.

### Example 9—Polydispersity Index of Polymer-Coated Nanoparticles

Cationic cores loaded with pDNA were coated at various w/w ratios with a PEG1k-PGA15k, PEG5k-PGA1.5k, PEG5k-PGA3.8, PEG5k-PGA7.5 or a PEG5k-PGA15k polymer coating to yield a polymer-coated nanoparticle. The polydispersity index was measured in 20 mM HEPES pH 7.2 and illustrated in FIG. 8. FIG. 8 further illustrates the data of a comparative cationic nanoparticle free of a polymer coating.

### Example 10—Comparative Example of Polydispersity Index of Coated Nanoparticles without a Neutral Segment

Cationic cores loaded with pDNA, similar to Example 9, were coated with a PGA7.5k or a PGA15k coating. The polydispersity index was measured in 20 mM HEPES pH 7.2 and illustrated in FIG. 8.

### Example 11—Polydispersity Index of Polymer-Coated Nanoparticles in Medium with Fetal Bovine Serum (FBS)

Cationic cores loaded with pDNA were coated at various w/w ratios with a PEG1k-PGA15k, PEG5k-PGA1.5k, PEG5k-PGA3.8, PEG5k-PGA7.5 or a PEG5k-PGA15k polymer coating to yield a polymer-coated nanoparticle. The polydispersity index was measured after 30 minutes incubation in 20 mM HEPES buffered culture medium (DMEM) with 10% (v/v) FBS. The results are illustrated in FIG. 9. FIG. 9 further illustrates the data of a comparative cationic nanoparticle free of a polymer coating.

### Example 12-Comparative Example of Polydispersity Index of Coated Nanoparticles without a Neutral Segment in a Medium with Fetal Bovine Serum (FBS)

Cationic cores loaded with pDNA, similar to Example 11, were coated at various w/w ratios with a PGA7.5k or a PGA15k coating polymer coating to yield polymer-coated nanoparticles. The polydispersity index was measured after 30 minutes incubation in 20 mM HEPES buffered culture medium (DMEM) with 10% (v/v) FBS. The results are illustrated in FIG. 9.

1. A polymer-coated nanoparticle comprising a biologically active payload, a cationic core and a polymer coating, wherein the polymer coating comprises a block copolymer comprising a polyanionic segment and a neutral segment, and wherein the cationic core comprises a poly(amido)amine cationic polymer, a poly(ester amine) cationic polymer and/or a quinoline-functionalized cationic polymer.

2. Polymer-coated nanoparticle according to claim 1, wherein the neutral segment comprises a non-charged and/or a zwitterionic component, wherein the neutral segment comprises poly(carboxybetaine acrylamide), poly(carboxybetaine methacrylamide), poly(sulfobetaine metacrylate), poly(methacryloyloxyethyl phosphoryl choline, polyvinylpyridiniopropanesulfonate), polyethylene glycol, polypropylene glycol, poly(glycerols) (PGs), poly(oxazolines) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), polysarcosine, polyacrylamide and poly(N-acryloylmorpholine) (PAcM) or a combination thereof, preferably polyethylene glycol.

3. Polymer-coated nanoparticle according to claim 1, wherein the polyanionic segment comprises polyglutamic acid, polyaspartic acid, polyacrylic acid or a combination thereof.

4. Polymer-coated nanoparticle according to claim 1, wherein the block copolymer is an A-B alternating polyblock copolymer, an A-B-A tri-block copolymer, a B-A-B tri-block copolymer or an A-B di-block copolymer wherein A denotes the one or more neutral segments and B denotes the one or more polyanionic segments.

5. Polymer-coated nanoparticle according to claim 1, wherein at least one of the neutral segments has a weight average molecular weight of at least 0.5 kDa.

6. Polymer-coated nanoparticle according to claim 1, wherein at least one of the polyanionic segments has a weight average molecular weight of at least 0.5 kDa.

7. Polymer-coated nanoparticle according to claim 1, wherein the weight ratio of the neutral segment to the polyanionic segment in the block copolymer is between 25:1 and 1:25, wherein the weight ratio is based on the weight average molecular weight of the segments.

8. Polymer-coated nanoparticle according to claim 7, wherein the weight ratio of the cationic polymer to the one or more polyanionic segments in the polymer-coated nanoparticles is less than 1:0.125, wherein the weight ratio is based on the weight average molecular weight.

9. Polymer-coated nanoparticle according to claim 1, wherein at least part the polyanionic segment is at least partially penetrating the bulk of the cationic core that is formed by the cationic polymer and/or wherein at least part of the neutral segment is at least partially protruding from the bulk of the cationic core that is formed by the cationic polymer.

10. Polymer-coated nanoparticle according to claim 1, wherein the polymer-coated nanoparticle has an average size of at most 350 nm, as determined by ISO 22412:2017.

11. Polymer-coated nanoparticle according to claim 1, wherein the polymer-coated nanoparticle has a zeta potential between-10 and 20 mV, as determined by ISO 13099-1.

12. Polymer-coated nanoparticle according to claim 1, wherein the zeta potential of the polymer-coated nanoparticle is at least 10 mV less than the zeta potential of a comparative cationic nanoparticle that is free from the polymer coating.

13. Polymer-coated nanoparticle according to claim 1, wherein the polymer-coated nanoparticle has a polydispersity index of less than 0.2, as determined by ISO 22412:2017.

14. Polymer-coated nanoparticle according to claim 1, wherein the polymer-coated nanoparticle has a polydispersity index of less than 0.2, in the presence of serum proteins, as determined by ISO 22412:2017.

15. Polymer-coated nanoparticle according to claim 1, wherein the polymer-coated nanoparticle has a polydispersity index in the presence of serum proteins is at least 0.05 less than the polydispersity index of a comparative cationic nanoparticle that is free from the polymer coating.

16. Polymer-coated nanoparticle according to claim 1, wherein the biologically active payload comprises oligonucleotides, polynucleotides, oligopeptides, polypeptides, proteins, small molecules or combinations thereof.

17. Polymer-coated nanoparticle according to claim 1 for use as a medicament, and/or for use as a therapeutic vaccine.

18. Method for forming the polymer-coated nanoparticle according to claim 1, wherein the method comprises combining the block copolymer, the payload and the cationic core in a liquid, wherein the cationic core and the block copolymer are combined in a weight ratio smaller than 1:0.125.

19. The polymer-coated nanoparticle according to claim 1 that acts as an adjuvant in a vaccine.
